(19) United States
(12) Patent Application Publication
(10) Pub. No.: US 2009/0024022 A1
(43) Pub. Date: Jan. 22, 2009

(54) SYSTEM AND METHOD FOR LYMPH NODE IMAGING USING CO-REGISTRATION OF CT AND MR IMAGERY

(75) Inventors: Fred S. Azar, Princeton, NJ (US); Mukesh G. Harisinghani, Lexington, MA (US); Ravi Seethamraju, Malden, MA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignees: Siemens Corporate Research, Inc., Princeton, NJ (US); Siemens Medical Solutions USA, Inc.; Massachusetts General Hospital Corporation

(21) Appl. No.: 11/830,231

(22) Filed: Jul. 30, 2007

Related U.S. Application Data
(60) Provisional application No. 60/854,182, filed on Oct. 25, 2006.

(51) Int. Cl. A61B 5/055

(52) U.S. Cl. 600/420

(57) ABSTRACT

A method for imaging lymph nodes includes acquiring first magnetic resonance (MR) image data pertaining to a subject prior to administration of lymphotropic nanoparticles. Second MR image data pertaining to the subject is acquired after administration of lymphotropic nanoparticles. The first and second MR image data are registered using non-rigid registration.

### CROSS-REFERENCE TO RELATED APPLICATION

The present application is based on provisional application Ser. No. 60/854,182, filed Oct. 25, 2006, the entire contents of which are herein incorporated by reference.

### BACKGROUND OF THE INVENTION

1. Technical Field

The present disclosure relates to imaging and, more specifically, to a system and method for lymph node imaging using co-registration of CT and MR imagery.

2. Discussion of the Related Art

The lymph nodes are components of the lymphatic system that act as filters. Lymph nodes have an internal honeycomb structure of reticular connective tissue that includes lymphocytes for fighting infection. Lymphatic metastases are small cancerous growths located in the lymph nodes. Lymphatic metastases are caused by cancerous cells that entered the lymphatic system and are thus spread to the lymph nodes.

The lymph nodes may be imaged to provide medical practitioners with the location of lymphatic metastases so that treatments such as radiotherapy and surgery may be finally targeted. For example, radiotherapy may be directed to the lymphatic metastases to provide more effective treatment and reduced side effects when compared to non-targeted systemic radiotherapy.

There are many available technologies for providing medical images of internal structures. For example, computed tomography (CT) imagery is a medical imaging technique that uses x-rays to image a set of two-dimensional slices. The slices are later combined to provide a three-dimensional representation of the internal structure being scanned.

Magnetic resonance (MR) imaging is a medical imaging technique that uses non-ionizing electromagnetic radiation to generate a set of two-dimensional slices. Like CT, MR image slices may be combined to provide a three-dimensional representation of the internal structure being scanned.

Accordingly, each imaging method has its own set of advantages and disadvantages. For example, the x-rays used in CT imagery are well suited for imaging tissue with relatively high atomic numbers, such as bone and calcifications and structures such as vessels and bowels. CT imagery may be enhanced by the use of radiocontrasts, such as barium sulfate, that may be injected or otherwise administered into bodily structures to provide added differentiation between the contrasted structure and the surrounding tissue.

MR imagery is better suited for non-calcified tissues, however, certain anatomical features and delineations may be more clearly resolved in CT imagery. In MR imagery, the structure to be imaged is subjected to a strong electromagnetic (EM) field. The EM field elevates the magnetic moment of the atomic nuclei of the scanned matter to an excited state and the rate in which the magnetic moments return to equilibrium (relax), a characteristic that is highly indicative of the particular form of matter being scanned, is closely monitored. By analyzing these relaxation times, the forms of matter within the scanned structure may be imaged.

There are several relaxation times that are relevant to MR imaging. For example, T1, also known as spin-lattice relaxation time, is defined as the component of relaxation which occurs in the direction of the ambient magnetic field. This generally comes about by interactions between the nucleus of interest and unexcited nuclei in the environment and ambient electric fields. T1 is measured as the time required for the magnetization vector to be restored to 63% of its original magnitude.

T2, also known as spin-spin relaxation time, is defined as the component of relaxation which occurs perpendicular to the ambient magnetic field. This relaxation is dominated by interactions between spinning nuclei that are already excited. T2 is measured as the time required for the transverse magnetization vector to drop to 37% of its original magnitude after its initial excitation.

T2* is the characteristic time constant that describes the decay of transverse magnetization, taking into account the inhomogeneity in static magnetic fields and the spin-spin relaxation in the human body. T2* is thus influenced by magnetic field gradient irregularities. T2* is increased with iron deposition.

### SUMMARY

A method for imaging lymph nodes includes acquiring first magnetic resonance (MR) image data pertaining to a subject prior to administration of lymphotropic nanoparticles. Second MR image data pertaining to the subject is acquired after administration of lymphotropic nanoparticles. The first and second MR image data are registered using non-rigid registration.

A system for imaging lymph nodes includes an MR database for providing first magnetic resonance (MR) image data pertaining to a subject prior to administration of lymphotropic nanoparticles. Second MR image data pertaining to the subject after administration of lymphotropic nanoparticles is also provided by the database. An image processing unit registers the first and second MR image data using non-rigid registration to provide combined image data. A display device displays the combined image data.

A computer system includes a processor and a program storage device readable by the computer system, embodying a program of instructions executable by the processor to perform method steps for imaging lymph nodes. The method includes acquiring first magnetic resonance (MR) image data pertaining to a subject prior to administration of FERUMOXTRAN-10. Second MR image data pertaining to the subject is acquired after administration of FERUMOXTRAN-10. Computed tomography (CT) image data pertaining to the subject is also acquired. The first and second MR image data are registered using non-rigid registration. The registered first and second MR image data is registered to the CT image data using non-rigid registration.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 is a flow chart showing a method for imaging lymph nodes according to an exemplary embodiment of the present invention;

FIG. 2 is a graph showing decreased average error distances for non-rigid registration when applied to exemplary embodiments of the present invention;

FIGS. 3(A)-(G) are sample images further illustrating the accuracy of non-rigid registration applied to exemplary embodiments of the present invention;

FIGS. 4(A)-(F) are sample images further illustrating the accuracy of non-rigid registration applied to exemplary embodiments of the present invention;

FIG. 5 is a sample image showing MR to CT registration according to an exemplary embodiment of the present invention; and

FIG. 6 shows an example of a computer system capable of implementing the method and apparatus according to embodiments of the present disclosure.

### DETAILED DESCRIPTION OF THE DRAWINGS

In describing the exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention may use FERUMOXTRAN-10 as a lymphotropic agent to image lymph node metastasis with magnetic resonance imaging (MRI). FERUMOXTRAN-10 is a contrast agent belonging to the class of USPIO (Ultra Small Superparamagnetic Iron Oxide) agents. FERUMOXTRAN-10 is lymphotropic in nature and has paved the way for MR in imaging lymph node metastasis. This highly sensitive and specific contrast agent may be used on many patients with cancers of the prostate, bladder, kidney and breast to identify metastases as small as 1 mm in lymph nodes that have become cancerous. When injected into the body, FERUMOXTRAN-10 is readily taken up by a type of white blood cells called macrophages which are found in abundance in the lymph nodes. The macrophages consume the FERUMOXTRAN-10 in a process called phagocytosis. Thus, FERUMOXTRAN-10 that enters healthy lymph nodes are readily taken up by macrophages and thus, FERUMOXTRAN-10 accumulates in the healthy lymph nodes. Diseased lymph nodes will tend to lack macrophages and as a result, FERUMOXTRAN-10 is not as likely to accumulate in diseased lymph nodes. Because FERUMOXTRAN-10 is readily detectable by MRI, healthy lymph nodes may be well imaged using MRI of a lymph nodes exposed to FERUMOXTRAN-10.

While the MRI of the lymph nodes exposed to FERUMOXTRAN-10 results in adequate imaging of healthy lymph nodes, diseased lymph nodes may be identified by comparing the image data of the MRI of lymph nodes exposed to FERUMOXTRAN-10 with image data of the lymph nodes showing the absence of FERUMOXTRAN-10. For example, pre-FERUMOXTRAN-10 contrast enhanced data may be acquired to provide a first MR image. Then FERUMOXTRAN-10 may be injected. Post-FERUMOXTRAN-10 contrast enhanced data may then be acquired to provide a second MR image. The first and second MR images may then be compared, for example, by a co-registration process, to identify diseased lymph nodes within an image.

Image registration techniques aim to specially align one image to another. This is accomplished by transforming different sets of data into one coordinate system. Image registration may either be rigid or non-rigid (elastic). In rigid image registration, linear transformations are applied to the data sets to achieve the desired transformation. Linear transformations may include translation, rotation, global scaling, shear and perspective components.

Non-rigid transformations allow for localized warping of image features and are thus able to effectively register images with local deformations. Non-rigid transformations may include polynomial wrapping, interpolation of smooth basis functions, and physical continuum models. Accordingly, non-rigid transformation may be better suited for medical image registration because of deformations and local warping that may occur. Examples of non-rigid transformation of medical images are described in U.S. Patent Application Publication Ser. No. 2005/0190189, which is hereby incorporated by reference.

The co-registered MR images may then be registered to a CT image. While the co-registered pre- and post-FERUMOXTRAN-10 images effectively identify diseased lymph nodes and/or lymphatic metastases, additional structural detail may be obtained from the CT. This additional structural detail may better allow medical practitioners to understand the location of the diseased lymph nodes in relation to other bodily structures. Accordingly, the co-registered MR images may be registered to the CT image, for example, by non-rigid transformation. Co-registration of images utilizing non-rigid transformation may be referred to herein as non-rigid registration.

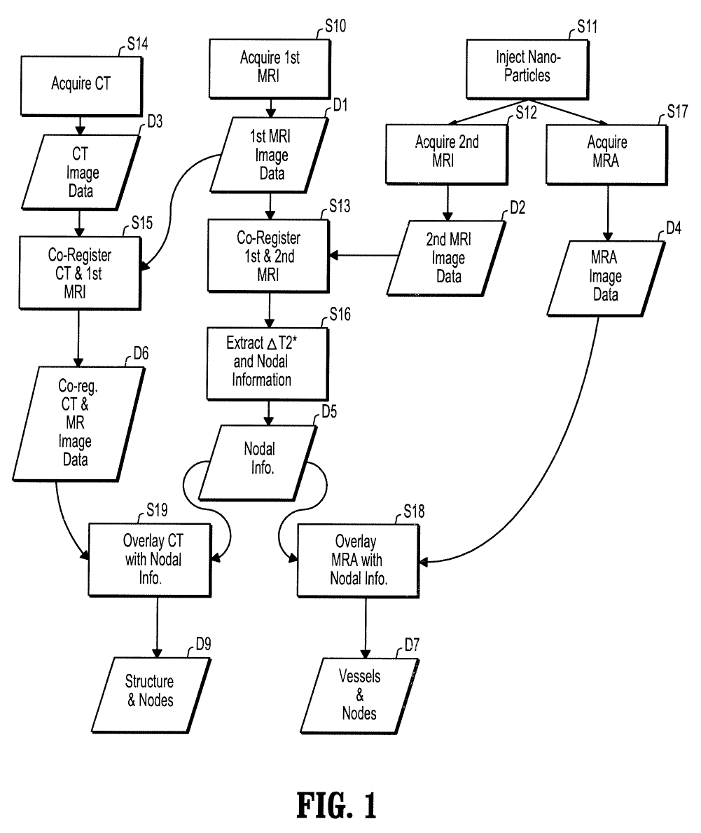

FIG. 1 is a flow chart showing a method for imaging lymph nodes according to an exemplary embodiment of the present invention. First magnetic resonance image data D1 may be acquired (Step S10). The acquired first MR image data D1 may be a reference MR image. In acquiring the first MR image data, T2 and T2* datasets may be acquired. MRI-visible contrast particles, for example, FERUMOXTRAN-10, may be administered (Step S11). Administration of the FERUMOXTRAN-10 may include injection of the particles, for example, suspended in a medium, into the vicinity of the lymph nodes, for example, into the lymphatic system or circulatory system. Then, second magnetic resonance image data D2 may be acquired (Step S12). In so doing, T2, T2* datasets may be acquired. The first MR image data D1 may therefore include both healthy and diseased lymph nodes, for example, with approximately equal intensity, while the second MR image data D2 may show the diseased lymph nodes with decreased intensity. The first MR image data D1 and second MR image data D2 may be co-registered, for example, using a non-rigid image registration technique (Step S13). Co-registration may include a combination of rigid and non-rigid image registration techniques.

The first MR image data D1 may provide structural information for all lymph nodes while the second MR image data D2 may provide enhanced information pertaining to diseased lymph nodes. By co-registering the two images, diseased lymph nodes may be identified on an image with structural information pertaining to diseased and healthy lymph nodes.

In co-registering the first and second MR images D1 and D2, the T2 dataset of the second MR image data D2 may be registered to the T2 dataset of the first MR image data D1. For example, the datasets may be aligned using a combination of rigid and/or non-rigid registration techniques. Similarly, the T2* dataset of the second MR image data D2 may be registered to the T2* dataset of the first MR image data D1. Accordingly, the datasets of the second MRI are aligned to the datasets of the first MRI. A difference image may then be obtained from the difference between the T2* data sets of the first and second MRIs (ΔT*) and nodal information D5 may be extracted (Step S16). The difference image may enable precise quantification of the extent of the metastasis infiltration into the lymph nodes.

Magnetic resonance angiography (MRA) image data D4 may be acquired (Step S17). The MRA image data D4 may be acquired independently or, for example, as part of the second MRI (Step S12). The MRA image data D4 may be acquired after injection of the nano-particles in Step S11. The MRA image data D4 may provide additional detail for the vessel tree structure of the lymphatic system, information that may be especially useful for performing surgery to removed diseased lymph nodes.

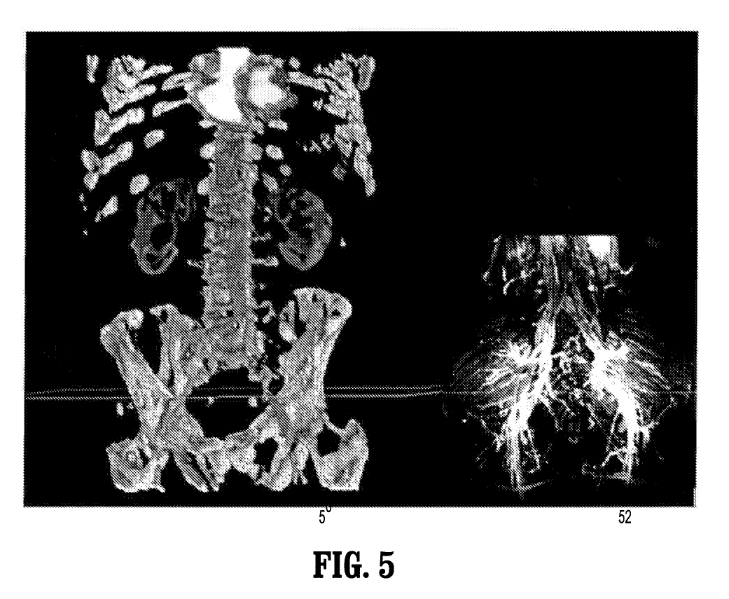

The MRA image data D4 may be overlaid with the nodal information D5 (Step S18). This overlay may provide an image D7 of the vessel tree with both diseased and healthy lymph nodes indicated thereon. An example of such an image D7 may be seen on FIG. 5 as image 52. Such an image D7 may be particularly useful for planning for surgery such as extraction of diseased lymph node tissue, for example, to minimize the area of dissection.

For added structural detail, CT image data D3 may be acquired prior to surgery or radiotherapy (Step S14). The CT image may then be co-registered with one or more of the MR images (Step S15) to provide co-registered CT and MR image data D6, for example, the CT image data D3 may be co-registered with the 1st MRI image data D1. Co-registration may be performed, for example, using a non-rigid registration technique. For example, co-registration may be performed using a combination of rigid and non-rigid registration techniques. Co-registration of the CT image may provide additional structural detail that is not included in the MR images.

The co-registered CT and MR image data D6 may then be overlaid with the nodal information D5 (Step S19). This overlay may result in an image showing structurally detailed CT data with both diseased and healthy lymph nodes indicated thereon (D9). An example of such an image D9 may be seen on FIG. 5 as image 50. Such an image D9 may be particularly useful, for example, for more precise radiotherapy planning by localizing the diseased lymph nodes in the pre-operative CT data. More precise planning may then lead to a more targeted radiotherapy, a reduced affected area and/or reduced side effects and morbidity.

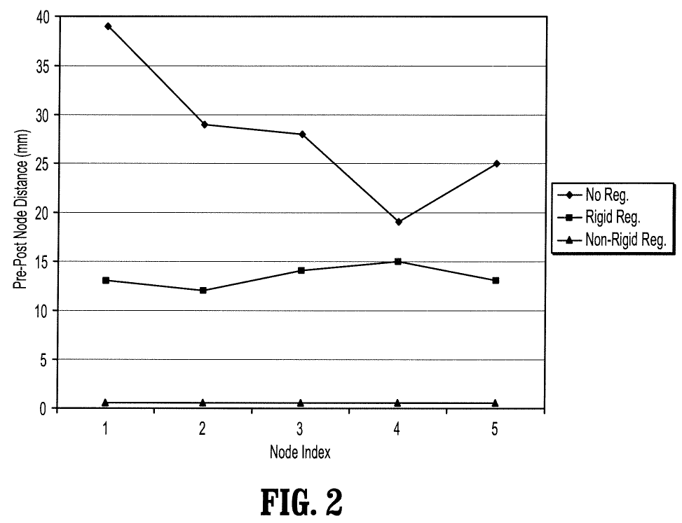

FIG. 2 is a graph showing decreased average error distances for non-rigid registration when applied to exemplary embodiments of the present invention. FIG. 2 relates to the co-registration (Step S13) of the pre-FERUMOXTRAN-10 MRI acquired in Step S10 with the post-FERUMOXTRAN-10 MRI acquired in Step S12. Without registration (shown as the top line with diamond points), there is a substantial error distance between the same nodes within a single coordinate system. When using rigid registration (shown as the middle line with square points), the average error distance drops from about 28 mm to about 13 mm. The non-rigid registration (shown as the bottom line with triangle points) yields a pixel accurate alignment of the lymph nodes.

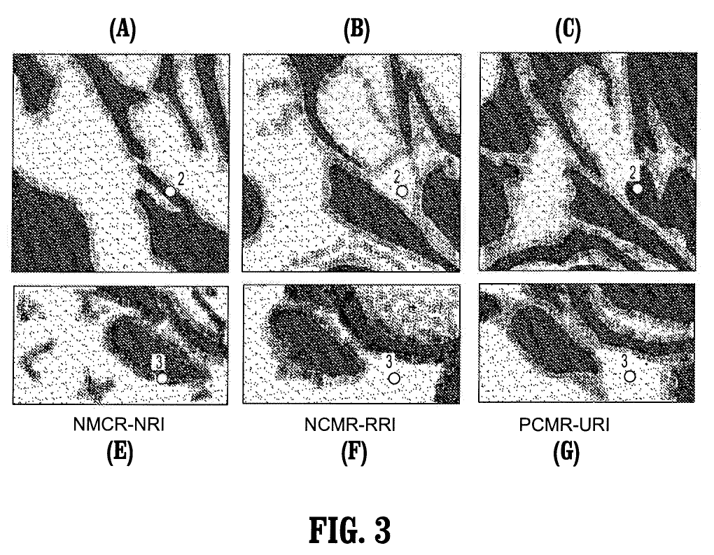

FIG. 3(A)-(G) are sample images further illustrating the accuracy of non-rigid registration applied to exemplary embodiments of the present invention. FIG. 3(C) is a sample MR image showing a lymph node 2 where FERUMOXTRAN-10 contrast has been introduced. Similarly, FIG. 3(G) is a sample MR image showing a lymph node 3 where FERUMOXTRAN-10 contrast has been introduced. FIG. 3(A) shows an image of the lymph node 2 prior to administration of the FERUMOXTRAN-10 contrast rigidly registered to the image of the lymph node 2 where FERUMOXTRAN-10 contrast has been introduced (FIG. 3(C)). Similarly, FIG. 3(E) shows an image of the lymph node 3 prior to administration of the FERUMOXTRAN-10 contrast rigidly registered to the image of the lymph node 3 where FERUMOXTRAN-10 contrast has been introduced (FIG. 3(G)). It can be seen from these figures that rigid registration of images before and after FERUMOXTRAN-10 contrast results in an image that lacks precision.

FIG. 3(B) shows an image of the lymph node 2 prior to administration of the FERUMOXTRAN-10 contrast non-rigidly registered to the image of the lymph node 2 where FERUMOXTRAN-10 contrast has been introduced (FIG. 3(C)). Similarly, FIG. 3(F) shows an image of the FERUMOXTRAN-10 contrast non-rigidly registered to the image of the lymph node 3 where FERUMOXTRAN-10 contrast has been introduced (FIG. 3(G)). It can be seen from these figures that non-rigid registration of images before and after FERUMOXTRAN-10 contrast results in a substantially precise image than when rigid registration is used.

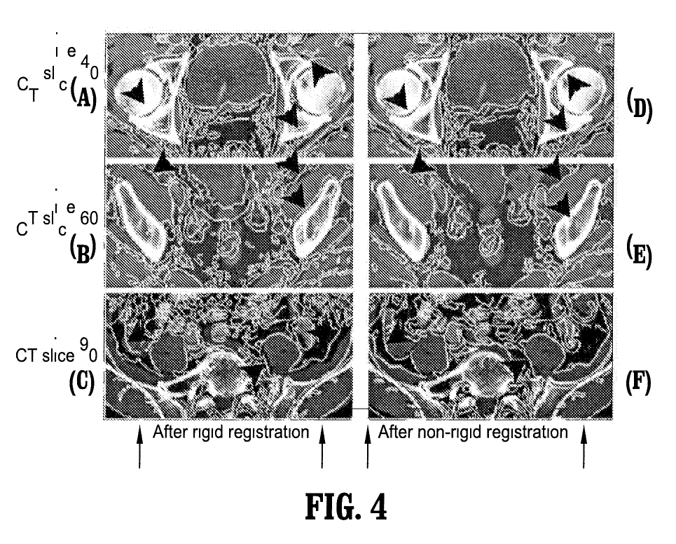

FIG. 4(A)-(F) are sample images further illustrating the accuracy of non-rigid registration applied to exemplary embodiments of the present invention. FIGS. 4(A),(B), and (C) show CT slices 40, 60, and 90 respectively, where the CT images have been rigidly registered against an MR image. FIGS. 4(D), (E), and (F) show CT slices 40, 60, and 90 respectively, where the CT images have been non-rigidly registered against an MR image. The arrows (appearing in black in FIGS. 4(A) and (D) and appearing in white in FIGS. 4(B), (C), (E), and (F)) show selected areas of clear alignment improvement in the non-rigidly registered images (FIGS. 4(D), (E), and (F)) compared to the rigidly registered images (FIGS. 4(A), (B), and (C)).

Using exemplary embodiments of the present invention, non-enlarged (occult) lymph nodes with small nodal metastases, for example, 5 mm and less, may be identified. A tumoricidal dose of radiotherapy may then be delivered to a well-defined target area and thus achieving optimum therapeutic ratio while minimizing the level of morbidity associated with radiotherapy.

FIG. 5 shows MR to CT registration according to an exemplary embodiment of the present invention. As described above with reference to FIG. 1, Step S15, the non-rigidly registered MR images may be registered to a CT image. Here, the MR image 52 is non-rigidly registered to the CT image 50 such that nodes from the MR image 52 are superimposed over the CT image 50.

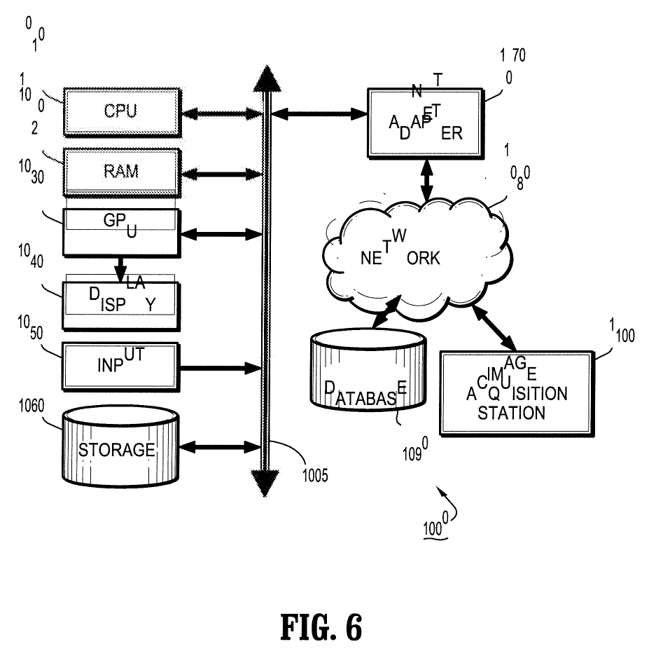

FIG. 6 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1010, random access memory (RAM) 1020, a graphical processing unit (GPU) 1030 connected to a display unit 1040, a network adapter 1070 connected to a network 1080, for example an intranet or the Internet, an internal bus 1005, and one or more input devices 1050, for example, a keyboard, mouse etc. As shown, the system 1000 may be connected to a data storage device 1060, for example, a hard disk.

The CPU 1010 may access and/or receive image data from an image acquisition station 1100 and/or a database 1090, for example, via the network 1080. The image acquisition station 1100 may include an MR scanner, a CT scanner or any other form of medical imaging device. The database 1090 may include previously acquired image data, for example, MR datasets and/or CT data sets.

The above specific exemplary embodiments are illustrative, and many variations can be introduced on these embodiments without departing from the spirit of the disclosure or from the scope of the appended claims. For example, elements and/or features of different exemplary embodiments may be combined with each other and/or substituted for each other within the scope of this disclosure and appended claims.

What is claimed is:

1. A method for imaging lymph nodes, comprising:
acquiring first magnetic resonance (MR) image data pertaining to a subject prior to administration of lymphotropic nanoparticles;
acquiring second MR image data pertaining to the subject after administration of lymphotropic nanoparticles; and
registering the first and second MR image data using non-rigid registration.

2. The method of claim 1, additionally comprising:
acquiring computed tomography (CT) image data pertaining to the subject; and
registering the registered first and second MR image data to the CT image data using non-rigid registration.

3. The method of claim 1, wherein the lymphotropic nanoparticles comprise FERUMOXTRAN-10.

4. The method of claim 1, wherein lymphatic metastases are targeted using the registered first and second MR image data.

5. The method of claim 2, wherein lymphatic metastases are targeted using the first and second MR image data registered to the CT image data.

6. The method of claim 1, wherein the acquiring of the first and second MR image data includes acquiring T2 and T2* datasets.

7. The method of claim 1, wherein the registering of the first and second MR image data further includes rigid image registration.

8. The method of claim 1, additionally comprising performing surgery or radiotherapy using the registered the first and second MR image data.

9. A system for imaging lymph nodes, comprising:
an MR database for providing first magnetic resonance (MR) image data pertaining to a subject prior to administration of lymphotropic nanoparticles and second MR image data pertaining to the subject after administration of lymphotropic nanoparticles;
an image processing unit for registering the first and second MR image data using non-rigid registration to provide combined image data; and
a display device for displaying the combined image data.

10. The system of claim 9, additionally comprising an MR scanner for acquiring the first and second MR image data and storing the first and second MR image to the MR database.

11. The system of claim 9, further comprising a CT database for storing computed tomography (CT) image data.

12. The system of claim 11, further comprising a CT scanner for acquiring CT image data and providing the CT image data to the CT database.

13. The system of claim 11, wherein the image processing unit registers the registered first and second MR image data with the CT image data stored in the CT database using non-rigid registration.

14. The system of claim 11, wherein the CT database and the MR database are the same database.

15. The system of claim 9, wherein the lymphotropic nanoparticles comprise FERUMOXTRAN-10.

16. The system of claim 9, further including:
an MR scanner for acquiring the first and second MR image data and storing the first and second MR image data in an MR database;
a CT scanner for acquiring CT image data and storing the CT image data in a CT database; and
a computer network for transmitting image data between the MR database, the CT database and the image processing unit.

17. A computer system comprising:
a processor; and
a program storage device readable by the computer system, embodying a program of instructions executable by the processor to perform method steps for imaging lymph nodes, the method comprising:
acquiring first magnetic resonance (MR) image data pertaining to a subject prior to administration of FERUMOXTRAN-10;
acquiring second MR image data pertaining to the subject after administration of FERUMOXTRAN-10;
acquiring computed tomography (CT) image data pertaining to the subject;
registering the first and second MR image data using non-rigid registration; and
registering the registered first and second MR image data to the CT image data using non-rigid registration.

18. The computer system of claim 17, wherein lymphatic metastases are targeted using the first and second MR image data registered to the CT image data

19. The computer system of claim 17, wherein the acquiring of the first and second MR image data includes acquiring T2 and T2* datasets.

20. The computer system of claim 17, wherein the method additionally comprises performing surgery or radiotherapy using the registered the first and second MR image data.
